(19) United States
(12) Patent Application Publication
(10) Pub. No.: US 2005/0130893 A1
(43) Pub. Date: Jun. 16, 2005

(54) Use of opioids in prevention of and recovery from a stress-induced crash in blood pressure

(76) Inventor: Joan Smith-Sonneborn, Laramie, WY (US)

Correspondence Address: DAVIS, BROWN, KOEHN, SHORS & ROBERTS, P.C.;THE FINANCIAL CENTER, 666 WALNUT STREET, SUITE 2500, DES MOINES, IA, 50309-3993, US

(21) Appl. No.: 10/953,637

(22) Filed: Sep. 29, 2004

Related U.S. Application Data
(60) Provisional application No. 60/507,234, filed on Sep. 30, 2003.
(60) Provisional application No. 60/539,144, filed on Jan. 26, 2004.

(51) Int. Cl. A61K 38/33

(52) U.S. Cl. 514/12; 514/17

(57) ABSTRACT

Receptor-selective peptide ligands, including deltorphin-D, DADLE, and DAGO, are used to prevent the occurrence of stress-induced blood pressure crash in mammals, to improve the likelihood and speed of recovery of mammals having experienced a crash in blood pressure, or to decrease mortality due to stress-induced crashes in blood pressure and eliminate or lessen the severity of the effects of blood pressure crashes. The invention is particularly suited to blood pressure crashes induced by heat and commonly known as heat stroke. If it is anticipated that the mammal will be susceptible to heat stroke, for example due to environmental temperatures, heat sensitivity of the mammal, or stress being experienced by the mammal, for example through physical labor or exercise, a pharmaceutically effective amount of one or more of the opioids is administered prior to the onset of heat stroke. Alternative or supplementary thereto, a pharmaceutically effective amount of one or more of the opioids is administered to a mammal after the onset of heat stroke.

This application claims priority to U.S. Patent Application Ser. No. 60/507,234, filed Sep. 30, 2003.

### BACKGROUND OF THE INVENTION

1. Field of the Invention

The invention relates generally to the use of receptor-selective peptide ligands in the prevention of and/or recovery from a stress-induced crash in blood pressure in mammals and, more specifically, to the administration of one or more of the opioids deltorphin D, DADLE, and DAGO to a mammal which may be susceptible to heat stroke prior to the onset of heat stroke and/or after the onset of heat stroke to prevent the occurrence of heat stroke and/or improve the recovery from heat stroke.

2. Background of the Art

Hibernation in bears shows cell structure damage like cells deprived of oxygen due to heart attack by blockage of an artery, hemorrhage, or muscle wasting by disuse. However, the bear maintains cell viability and muscle strength quickly lost in humans by inactivity. Studies by Dr. Peter Oeltgen and colleagues at the University of Kentucky have tried to isolate the “Hibernation Inducing Trigger” (HIT) and led to the discovery of an opioid like sequence that has been fully characterized and is referred to herein as deltorphin D. Deltorphin D is a linear 17 amino acid peptide which has been synthesized on a commercial peptide synthesizer by the University of Kentucky. Deltorphin D has the amino acid sequence Tyr-D-Ala-Phe-Ala-Asp-Val-Ala-Ser-Thr-Ile-Gly-Asp-Phe-Phe-His-Ser-Ile-NH2 and a MW of 1860.1 with alanine as D-isomer. In addition, the dried skin of Pyllomedussa bicolor “sapo” has been found to contain deltorphin opioids and has been used by the Matses Indians of Peru for induced resistance to hunger, thirst, danger, and increased strength. The deltorphin opioids are among the peptides found related both in the hibernation trigger and in the frog skin.

Deltorphin D has been identified as a treatment for ischemia in mammals (U.S. Pat. No. 6,544,950). Deltorphin D is reported as useful for treating cerebral ischemia and ischemic heart disease. Although its specific mode of action on ischemia is unknown, deltorphin D is asserted to exhibit a specific and reproducible effect on decreasing neurological deficit and cerebral infarct volume (Ibid.).

DADLE is a delta opioid agonist [D-Ala2-D-Leu5]enkephalin acetate (Tyr-D-Ala-Gly-Phe-D-Leu). DADLE has been shown to increase survival of hemorrhagic shock and quickly reverses fallen mean arterial pressure. Summers, R. L., Li, Z., and Hildebrandt, D. Effect of a Receptor Agonist on Duration of Survival During Hemorrhagic Shock, Acad. EmergMed. 2003; 10: 587-593. In addition, DAGO, a mu opioid agonist [D-Ala2-MePhe4-Glyol]enkephalin (Tyr-D-Ala-Gly-Phe-D-Leu) was shown to restore mean arterial pressure after hemorrhage but not DADLE when the bleeding was non-lethal. Feuerstein, G., Powell, E., and Faden, A. I. Central Effects of Mu, Delta, and Kappa Receptor Agonists in Hemorrhagic Shock. Peptides. 1985; 6 Suppl 1: 11-3.

### SUMMARY OF THE INVENTION

The invention consists of the use of the opioids deltorphin D, DADLE, and DAGO to prevent the occurrence of stress-induced blood pressure crash in mammals, to improve the likelihood and speed of recovery of mammals having experienced a crash in blood pressure, and to decrease mortality due to stress-induced crashes in blood pressure and eliminate or lessen the severity of the effects of blood pressure crashes. The invention is particularly suited to blood pressure crashes induced by heat and commonly known as heat stroke. If it is anticipated that the mammal will be susceptible to heat stroke, for example due to environmental temperatures, heat sensitivity of the mammal, or stress being experienced by the mammal, for example through physical labor or exercise, a pharmaceutically effective amount of one or more of the opioids is administered prior to the onset of heat stroke. Alternative or supplementary thereto, a pharmaceutically effective amount of one or more of the opioids is administered to a mammal after the onset of heat stroke.

An object of the invention is to provide a method of preventing stress-induced blood pressure crash in mammals.

A further object of the invention is to provide a method of preventing heat stroke in mammals.

Another object of the invention is to provide a method of ameliorating the effects of stress-induced blood pressure crash, particularly heat stroke, in mammals.

These and other objects of the invention will become apparent to those of skill in the art upon a review of this specification and the associated drawings.

### DETAILED DESCRIPTION OF A PREFERRED EMBODIMENT

Experiment 1

Experiments were initiated to determine if pretreatment in living rats with deltorphin could alter the stress response to surgically induced heart attack. Prior work had shown that the stress response is altered using deltorphin D. The continued study of the molecular stress response to pre- or post-treatment of hemorrhagic animals to controlled bleeding was of interest.

The present invention centers on the concept of using opioids as a protective agent against heat shock. Earlier work studied how two opioids, deltorphin D and DADLE would affect molecular stress response to heat and survival. These observations indicated that, in males, Deltorphin D lowered body temperature and was protective against heat, while DADLE did not show these effects. The current study focused on the use of deltorphin D to protect against heat stroke by administration before, after, or before and after, heat stroke. Heat stroke is defined as a rapid rise in blood pressure followed by a crash in pressure in response to overheating.

Male rats were used in the study. The first rat weighed approximately 500 g and was anesthetized with ketamine and then wrapped in a heating blanket to raise its body temperature and induce heat stroke. The temperature and blood pressure of the anesthetized rat was monitored with a recording colonic temperature probe and a tail blood pressure cuff. When the symptoms of heat stroke were observed, 1.8 ml of 1.0 mg/ml pure deltorphin D was administered in the tail vein. The amount of deltorphin administered was based on the earlier hemorrhagic studies of Dr. Oeltgrn. The rat survived only a few minutes. It was believed that administration of the deltorphin in the tail vein did not allow for a sufficient rate of uptake of the deltorphin by the animal to raise blood levels sufficiently high sufficiently quickly to achieve the protective or ameliorative effect.

The second male rat was catherized with an arterial line two weeks previous to the next experiment to provide a means for more rapidly providing the deltorphin. The second rat weighed approximately 500 g and was anesthetized with ketamine and then wrapped in a heating blanket set at 51.5° C. to raise its body temperature and induce heat stroke. Prior to being wrapped in the heating blanket, a pre-treatment injection of 1.8 ml of 1.0 mg/ml pure deltorphin was provided through the catheter. The temperature and blood pressure of the anesthetized rat was monitored with a recording colonic temperature probe and blood pressure was monitored and recorded off of the arterial line. The blood pressure of the animal was observed to rise rapidly and then crash when its body temperature had reached 41° C., indicating the onset of heat stroke. One hour later, another 1.8 ml of 1.0 mg/ml pure deltorphin was administered through the catheter. At that time, the rat was having difficulty moving. One hour later, or two hours after the onset of heat stroke, the rat was able to move some on its own. Approximately five hours later, or seven hours after the onset of heat stroke, the animal was able to drink 5 ml of water out of a pipette. The animal remains alive and healthy more than four weeks after the test.

A third male rat weighing approximately 200 g was catherized with an arterial line the day before the test. The third rat was anesthetized with ketamine and then wrapped in a heating blanket set at 51.5° C. to raise its body temperature and induce heat stroke. Prior to being wrapped in the heating blanket, a pre-treatment injection of 0.7 ml of 1.0 mg/ml pure deltorphin was provided through the catheter. The temperature and blood pressure of the anesthetized rat was monitored with a recording colonic temperature probe and blood pressure was monitored and recorded off of the arterial line. The blood pressure of the animal was observed to rise rapidly and then crash when its body temperature had reached 41° C., indicating the onset of heat stroke. Fifteen minutes later, another 0.7 ml of 1.0 mg/ml pure deltorphin was administered through the catheter. The rat died approximately 75 minutes after the onset of heat stroke. While the third rat did not survive as long as the second rat, it is believed that the installation of the arterial line catheter only the day before the test left the rat in a weakened condition and more susceptible to fatal heat stroke. Nonetheless, the third animal lived for 75 minutes. This survival rate is long when compared to the study reported at Chi WT, Kao TY, Lin MT. Neurosci Res. 1996 24:159-163 wherein rats in which heat stroke was induced lived only about 17 minutes after the onset of heat stroke and continuous perfusion of the animals with saline did nothing to extend their survival. Given the weakened condition of the third rat and its survival for more than four times the expected time in the absence of deltorphin administration, the deltorphin appears to have had a significant effect.

Experiment 2

Heat shock proteins (HSPs) and ubiquitin are up-regulated in response to many types of stress, including heat shock, hypoxia, ischemia, and oxidative damage. HSP70 and ubiquitin have been shown to prevent cellular damage during times of stress by repairing denatured proteins and degrading proteins that are too damaged to be repaired. Additionally, the heat shock response is a classic example of induced gene expression. Even mild heat shock initiates a heat shock response characterized by increased synthesis of heat shock proteins. The HSPs confer increased tolerance to high temperatures and also to other physiological stresses, such as hypoxia or ischemia.

Experiments were undertaken to study the molecular stress response after heat stress with and without opiate pretreatment in rats. Since it is know that both behavior and pain response to opiates are gender-dependent, both male and female rats were used. The animals were treated by exposure to dry heat at a colonic temperature of 44° C. The animals were sacrificed and brain, leg and heart tissue was removed. The molecular stress responses chosen were HSP 70 and ubiquitin. HSP 70 is a chaperone protein which helps repair damaged proteins, and UbB and UbC forms of ubiquitin are induced by cell damage. Messenger RNA was extracted and analyzed using Northern Blot determination of transcript intensity in saline heat shock controls versus pretreatment with DADLE, a non-specific delta opioid agonist, and deltorphin D.

The subjects were male and female Charles River rats. The rats were divided by sex and by treatment groups. There were four treatment groups: control saline treatment, heat shock-saline treatment, heat shock-DADLE treatment, and heat shock-deltorphin-D treatment. The rats underwent a three-day protocol, receiving injections each day and on the final day undergoing heat shock. The final treatment was given on day three of the experiment, 20 minutes before heat shock. Treatment consisted of intravenous tail injections at 1 mg/kg body weight for all groups. Heat shock was initiated by 1 hour exposure in an incubator at 44° C. The rats were sacrificed 2 hours later and alteration of molecular responses monitored by gel electrophoresis of RNA using Northern blots. DNA sequences for the molecular markers HSP70 and ubiquitin were used to probe the blots. The probes were radioactively labeled with p³² to label the probe. When the probe “finds” its complementary sequence, it binds to it and the intensity of the signal which results is determined using Molecular Analyst software of the signals on X-ray film. The intensity of the black areas on the film is a measure of gene activity altered by treatment with the respective opioids.

Table 1 displays the measurements of UbB, UbC, and HSP70 taken from the brain, leg, and heart tissues of the animals.

TABLE 1
Measurements of UbB, UbC, and HSP70 of brain,
leg, and heart tissues

| | | UBC | UBB | HSP |
|---|---|---|---|---|
| Female Brain | Control | 1.201473 | 1.2836 | 0.681472 |
| | Saline | 2.627627 | 3.677659 | 1.29422 |
| | DADLE | 0.799339 | 1.688369 | 1.294665 |
| | Delt D | 0.766367 | 2.844331 | 0.026339 |
| Male Brain | Control | 0.19688 | 0.266868 | 0.2292 |
| | Saline | 1.533722 | 3.426267 | 1.656027 |
| | DADLE | 2.355863 | 4.596134 | 1.390545 |
| | Delt D | 1.693696 | 2.258625 | 1.043482 |
| Female Leg | Control | 1.106478 | 1.565192 | 0.796453 |
| | Saline | 1.388025 | 3.130829 | 1.443186 |
| | DADLE | 0.899518 | 2.760435 | 0.884035 |
| | DeltD | 0.243936 | 2.803201 | 0.900055 |
| Male Leg | Control | 0.090675 | 1.323051 | 0.522361 |
| | Saline | 0.217358 | 0.860751 | 0.349456 |
| | DADLE | 0.179993 | 0.600468 | 0.329367 |
| | DeltD | 0.133925 | 2.319173 | 0.669663 |
| Female Heart | Control | 0.586292 | 1.351269 | 0.40009 |
| | Saline | 0.453312 | 3.861291 | 1.164585 |
| | DADLE | 0.560477 | 3.570157 | 0.895848 |
| | DeltD | 0.859509 | 2.844996 | 1.26996 |
| Male Heart | Control | 0.747064 | 1.245747 | 0.747284 |
| | Saline | 0.628474 | 1.450555 | 0.594243 |
| | DADLE | 0.583698 | 0.775377 | 0.536446 |
| | DeltD | 0.043452 | 0.561333 | 0.369674 |

The differences in intensity of the mRNA signals for the different groups of animal are shown in Table 2.

TABLE 1
Intensity of mRNA signals by group

| Females | | | | Males | | |
|---|---|---|---|---|---|---|
| | Saline | DADLE | Deltorphin-D | Saline | DADLE | Deltorphin-D |

BRAIN:

| | | | | | | |
|---|---|---|---|---|---|---|
| UBC | 0.012*▴ | 0.7 | 0.7 | 0.276 | 0.109 | 0.074 |
| UBB | 0.003*▴ | 0.61 | 0.101 | 0.009*▴ | 0.005*▴ | 0.045*▴ |
| HSP | 0.026*▴ | 0.108 | 0.137 | 0.002*▴ | 0.038*▴ | 0.028*▴ |
| LEG: | | | | | | |
| UBC | 0.299 | 0.95 | 0.001*▾ | 0.005*▾ | 0.004*▾ | 0.005*▾ |
| UBB | 0.756 | 0.052 | 0.652 | 0.306 | 0.231 | 0.803 |
| HSP | 0.747 | 0.209 | 0.831 | 0.18 | 0.152 | 0.421 |
| HEART: | | | | | | |
| UBC | 0.51 | 0.859 | 0.3 | 0.835 | 0.439 | 0.005*▾ |
| UBB | 0.038*▴ | 0.001*▴ | 0.009*▴ | 0.124 | 0.718 | 0.426 |
| HSP | 0.019*▴ | 0.008*▴ | 0.000*▴ | 0.812 | 0.211 | 0.047*▾ |

*denotes significance (p < 0.05)
▴ (▾) indicate an increase (decrease) compared to saline control

Deltorphin D and DADLE pretreatments resulted in significant (p<0.05) alteration of the molecular stress response in drug-unique patterns. Ubiquitin expression was found to be specific for ubiquitin transcript size (UbB vs. UbC), tissue, and sex. DADLE, not deltorphin D, pretreatment, sensitized male rats to heat. Table 3 shows the change in body core temperature (BT) with saline, DADLE, and deltorphin-D injections.

TABLE 3
Changes in Body Temperature

| | Temperature Day 1 | Temperature Day 2 |
|---|---|---|
| Saline Female 1 | −0.95 | −0.2 |
| Saline Female 2 | −0.5 | −2.35 |
| Saline Female 3 | 0.05 | −3 |
| Saline Female 4 | −0.15 | 0.45 |
| DADLE Female 1 | −0.85 | −0.35 |
| DADLE Female 2 | −2.4 | −3.45 |
| DADLE Female 3 | 0.45 | −1.65 |
| DADLE Female 4 | −1.85 | −1.45 |
| Deltorphin-D Female 1 | −0.7 | −1.4 |
| Deltorphin-D Female 2 | −0.7 | −1.7 |
| Deltorphin-D Female 3 | 0.2 | −1.6 |
| Deltorphin-D Female 4 | −1.6 | −1.9 |
| Deltorphin-D Female 1 | −1.5 | −2.8 |
| Saline Male 1 | 0.34 | 0.5 |
| Saline Male 2 | 0.14 | −0.75 |
| DADLE Male 1 | 2.36 | 1.55 |
| DADLE Male 2 | 1.82 | 0.2 |
| Deltorphin-D Male 1 | −2.6 | −2.9 |
| Deltorphin-D Male 2 | −2.2 | −1.4 |
| Deltorphin-D Male 3 | −1.2 | −0.7 |
| Deltorphin-D Male 4 | −3.1 | −2.2 |

Changes in body temperature in the females were not significant, whereas all of the changes in body temperature in the males were significant on Day 1 and the difference between DADLE and deltorphin-D animals on Day 2 males was also significant. DADLE raises body temperature and deltorphin-D lowers body temperature significantly. It was concluded from the study that there are tissue and sex specific differences in response to heat shock and opiate injections in the rat and that females appear to be less sensitive than males to both.

While no accepted explanation for the activity of deltorphin exists, one possible consideration may be that the pre-administration of deltorphin actually lowers the temperature at which the animal enters heat stroke. Evidence exists that qualitatively different physiological changes occur in rats when their body temperature reaches 43° C., changes which may not allow the animal to survive. If the onset of heat stroke begins at a lower temperature in treated rats, perhaps the animal is more likely to survive because the irreversible, or difficult to reverse processes at higher temperatures have not yet occurred or at least accumulated to a lethal level. This effect may assist in the prevention of and recovery from heat stroke together with the effects of deltorphin on cell damage due to oxygen deprivation.

Although only a few exemplary embodiments of this invention have been described in detail above, those skilled in the art will readily appreciate that many modifications are possible in the embodiments given without materially departing from the novel teachings and advantages of this invention. Accordingly, various modifications, adaptations, and combinations or various features of the described embodiments can be practiced without departing from the scope of the invention as set forth in the claims.

1. A method for ameliorating the effects of stress-induced blood pressure crash in mammals, comprising the step of administering a therapeutically effective amount of one or more opioids selected from the group consisting of deltorphin D, DADLE and DAGO to the mammal at a time after the crash.

2. A method for protecting a mammal against stress-induced blood pressure crash, comprising the step of administering a therapeutically effective amount of one or more opioids selected from the group consisting of deltorphin D, DADLE and DAGO to the mammal at a time prior to the crash.

3. A method as defined in claim 2 or claim 3 wherein the blood pressure crash is due to heat stroke.

4. A method as defined in claim 1 wherein the opioid is administered between about zero minutes and about 180 minutes after the crash in blood pressure.

5. A method as defined in claim 2, wherein the opioid is administered between about 180 minutes and about zero minutes before the crash in blood pressure.

6. A method as defined in claim 1 or claim 2 wherein the amount of opioid is between about 0.1 mg and about 5 mg per kilogram in a volume adjusted for body weight.
